(19) United States
(12) Patent Application Publication
(10) Pub. No.: US 2016/0324761 A1
(43) Pub. Date: Nov. 10, 2016

(54) Method of Making A Hair And Skin Conditioning Composition

(71) Applicant: Joseph Bell, Alto, GA (US)

(72) Inventor: Joseph Bell, Alto, GA (US)

(21) Appl. No.: 14/706,949

(22) Filed: May 7, 2015

(51) Int. Cl. A61K 8/97; A61Q 19/00; A61K 8/92; A61Q 5/12

(52) U.S. Cl. CPC A61K 8/97; A61Q 5/12; A61Q 19/00; A61K 8/922; A61K 2800/805

(57) ABSTRACT

A method of making a hair and skin conditioning composition includes the steps of placing walnuts in water to define a mixture. Boiling the mixture for at least three hours and straining the mixture to remove the walnuts and retain a fluid provided after the straining of the mixture. The fluid may then be used for conditioning a person's skin and hair.

### BACKGROUND OF THE DISCLOSURE

### Field of the Disclosure

The disclosure relates to hair and skin conditioners and more particularly pertains to a new hair and skin conditioner for protecting and promoting healthy hair and skin.

### SUMMARY OF THE DISCLOSURE

An embodiment of the disclosure meets the needs presented above by generally comprising the steps of placing walnuts in water to define a mixture. Boiling the mixture for at least three hours and straining the mixture to remove the walnuts and retain a fluid provided after the straining of the mixture. The fluid may then be used for conditioning a person's skin and hair.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### DESCRIPTION OF THE PREFERRED EMBODIMENT

A new hair and skin conditioner embodying the principles and concepts of an embodiment of the disclosure will be described.

As best stated herein, the method of making a hair and skin conditioning composition generally comprises the boiling of walnuts for several hours, recovering a fluid therefrom and possibly adding a fatty material (or oil) to the fluid depending on its usage. The fluid, with or without the fatty material, is used for conditioning a person's skin and hair and would more typically include the fatty material to form a final composition to be used on a person's skin.

More particularly, the method includes placing walnuts in water to define a mixture such that a ratio of walnuts to water by weight is between 1 to 30 and 1 to 45. Though the water will typically be weighed by fluid ounces as opposed to avoirdupois ounces, it is assumed that the similarity between these two measurements for water is such that any differences in final measurements will be irrelevant to the practicing of the method herein since the method may be practiced such that generally 1.0 ounce of walnuts is used with approximately 36.5 fluid ounces of water+/−5.0% of the water. The walnuts may more particularly include black walnuts grown on walnut trees, Juglans nigra, which are shelled and mixed with water such that approximately 50 whole black walnuts is mixed with 2.0 gallons of water. This mixture is brought to a boil and boiled for at least three hours and may be boiled for up to four hours until approximately between 20% and 30% of the water has evaporated. The mixture is then strained to remove any walnut or walnut particles while the fluid remaining after the straining is retained. In the above example, approximately 1.5 gallons of fluid will remain. This fluid may be bottled in this state and used for hair conditioning purposes and more particularly for preventing split ends, softening the hair, reducing frizzing of hair and to prevent dandruff.

To the retained fluid may be added a fatty material such that a composition is defined which may be more suitable for usage on a person's skin, though the composition may also be used for the person's hair. In particular, the fatty material is added to the fluid such that a ratio of the fatty material to the fluid is between 1 to 50 and 1 to 75 by volume. More specifically, the ratio may be approximately 1.0 fluid ounces of fatty material to 64.0 fluid ounces of the fluid. The fatty material may comprise any number of materials, however, it has been found that shea butter extracted from nuts of the shea tree (Vitellaria pardoxa), or coconut oil from coconuts harvested from the coconut palm (Cocos nucifera) work particular well for skin conditioning purposes.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

1. A method of making a skin and hair ointment, said method including the steps of:
placing shelled walnuts in water to define a mixture;
boiling the mixture for at least three hours; and
straining the mixture to remove the walnuts and retaining a fluid provided after the straining of the mixture.

2. The method of making a skin and hair ointment according to claim 1, wherein said method further includes the step of adding a fatty material to said fluid such that a ratio of said fatty material to said fluid is between 1 to 50 and 1 to 75.

3. The method of making a skin and hair ointment according to claim 1, wherein said fatty material comprises shea butter or coconut oil.

4. The method of making a skin and hair ointment according to claim 1, wherein the step of placing said walnuts in said water includes a ratio of walnuts to water by weight being between 1 to 30 and 1 to 45.

5. The method of making a skin and hair ointment according to claim 1, wherein the step of boiling the mixture includes said mixture being boiled until between 20% and 30% of the water has evaporated.

6. A method of making a skin and hair ointment, said method including the steps of:
placing shelled walnuts in water to define a mixture such that a ratio of walnuts to water by weight is between 1 to 30 and 1 to 45;
boiling the mixture for at least three hours until between 20% and 30% of the water has evaporated;
straining the mixture to remove the walnuts and retaining a fluid provided after the straining of the mixture; and
adding a fatty material to said fluid such that a ratio of said fatty material to said fluid is between 1 to 50 and 1 to 75, said fatty material comprising shea butter (shea tree nut Vitellaria pardoxa) or coconut oil £Cocos nucifera).
